(19) United States
(12) Patent Application Publication
(10) Pub. No.: US 2015/0018743 A1
(43) Pub. Date: Jan. 15, 2015

(54) Nail and Nail Bed Protecting Bandage

(71) Applicant: James Detwiler, Kutztown, PA (US)

(72) Inventor: James Detwiler, Kutztown, PA (US)

(21) Appl. No.: 13/941,506

(22) Filed: Jul. 14, 2013

(51) Int. Cl. A61F 13/10; A61F 13/06; A61F 13/02

(52) U.S. Cl. CPC A61F 13/105; A61F 13/02; A61F 13/068; A61F 2013/00272
USPC 602/58

(57) ABSTRACT

A combined bandage having an attachment portion for overlying and circumscribing a human digit with an injured nail and nail bed that is configured with a plurality of extensions to wrap around the injured digit and secure the bandage in place. A protective portion sized to overlie the injured nail and nail bed and adhered to the attachment portion of the bandage for providing a solid reinforcing surface to protect the injured nail and nail bed from further injury through direct force, pressure or incidental striking.

### BACKGROUND OF THE INVENTION

Nail and nail bed injuries are quite common in the workplace and are becoming more common as people exercise more and participate in sporting activities. These injuries also occur to those do-it-yourselfers who are attempting to perform jobs that they do not normally perform in their daily lives resulting in an injury.

Injuries to the nails, and to the underlying nail beds, are often involved with injuries to the fingers or toes. These injuries may range from a subungual hematoma due to a sharp blow or pressure to the end of the finger or toe on the surface of the nail to the tearing of the nail or the removal of the entire nail leaving the nail bed exposed. Nails grow slowly with self-repair and regrowth delayed for sometimes one month following the injury. In comparison, a normal, uninjured nail may take as long as 100 days to reach full length to the end of the respective digit. During the regrowing process, the nail and nail bed need to be protected from further injury and damage.

A human nail consists of several parts: a nail plate or body, the hard covering of the end of the finger or toe; a nail bed, the skin directly under the nail plate made up of dermis and epidermis with blood vessels and nerves running throughout the nail bed; and a nail matrix, the portion or root at the base of the nail under the cuticle that produces new cells for the nail plate and determines the shape and thickness of the nail. If the nail plate is torn or ripped away in an injury, the nail bed may be damaged as well. The nail bed must be protected and the nail matrix must be allowed to generate new cells without impediment on the nail bed. The best method to do this is to cover the nail bed with a bandage that is sufficiently thick to protect the nail bed, but which prevents the full use of the finger or toe. This present level of treatment may be coupled with a splint to inhibit the finger or toe from bending to also aid in the healing process. In treating a nail and/or nail bed injury in the described fashion the injured digit is substantially rendered unusable and, if needed for work, other duties may be required before returning to full use of the digit.

It is, therefore, an object of the present invention to provide a reinforced section of the bandage to prevent additional direct force damage to the regrowing nail and nail bed following a nail tearing or compression injury to the digit. Another object of the present invention is to combine in a single bandage both a wound dressing and a protective section to prevent further injury without impeding the use of the digit in daily activities. It is a still further object of the present invention to structure the combination bandage such that the reinforced section directly overlies the injured nail and nail bed without impeding the use of the digit for daily activities.

Other objects will appear hereinafter.

### SUMMARY OF THE INVENTION

A different type of specialty wound dressing is provided for injured nails that are torn or entirely ripped away from human digits. This combination bandage provides both a wound dressing and a solid reinforcing and protective covering on the outer surface of the bandage to protect the injured nail and nail bed from further injury that may occur in the daily life of the injured person. This is accomplished without the need for splinting the digit or providing excessive bandaging virtually rendering the digit non-useable while bandaged or splinted.

A combined bandage is configured with an attachment portion for overlying and circumscribing a human digit that has an injured nail and nail bed. The attachment portion of the combined bandage is configured with a plurality of elongate extensions sufficient to wrap around the injured digit and secure the combined bandage in place. The combined bandage also has a wound dressing portion attached to and centered on the underside of the attachment portion for contacting the injured nail and nail bed as in conventional bandages and dressings. However, the combined bandage is different from the conventional bandages in that on its outer surface a protective portion that is sized to overlie the injured nail and nail bed is adhered to an adhesive pad that is attached to and centered on the top side of the attachment portion and is capable of receiving and retaining the protective portion in position. The protective portion is designed to provide a solid reinforcing surface to protect the injured nail and nail bed from further injury through direct force, pressure or incidental striking.

The protective portion combined bandage not only overlies the injured nail and nail bed, but is dimensioned to extend outward in all directions to contact non-injured portions of the injured digit. The protective portion of the combined bandage is also configured and colored to resemble a human finger or toe nail. The combined bandage is selectively dimensioned such that the overall size of the combined bandage is variable to proportionately fit differently sized human digits.

### BRIEF DESCRIPTION OF THE DRAWINGS

For the purpose of illustrating the invention, there is shown in the drawings forms which are presently preferred; it being understood, however, that the invention is not limited to the precise arrangements and instrumentalities shown.

FIG. 1 is an isometric view of the reinforced protective bandage of the present invention.

FIG. 2 is an exploded view of the reinforced protective bandage of the present invention showing the protective section and interposed adhesive layer above the bandage.

FIG. 3A is a plan view of an extra-large sized version of the reinforced protective bandage of the present invention.

FIG. 3B is a plan view of a large sized version of the reinforced protective bandage of the present invention.

FIG. 3C is a plan view of a medium sized version of the reinforced protective bandage of the present invention.

FIG. 3D is a plan view of a small sized version of the reinforced protective bandage of the present invention.

FIG. 4 is a side view of the reinforced protective bandage of the present invention in position about a finger having an injured nail and nail bed.

### DETAILED DESCRIPTION OF THE PREFERRED EMBODIMENTS

The following detailed description is of the best presently contemplated mode of carrying out the invention. The description is not intended in a limiting sense, and is made solely for the purpose of illustrating the general principles of the invention. The various features and advantages of the present invention may be more readily understood with reference to the following detailed description taken in conjunction with the accompanying drawings.

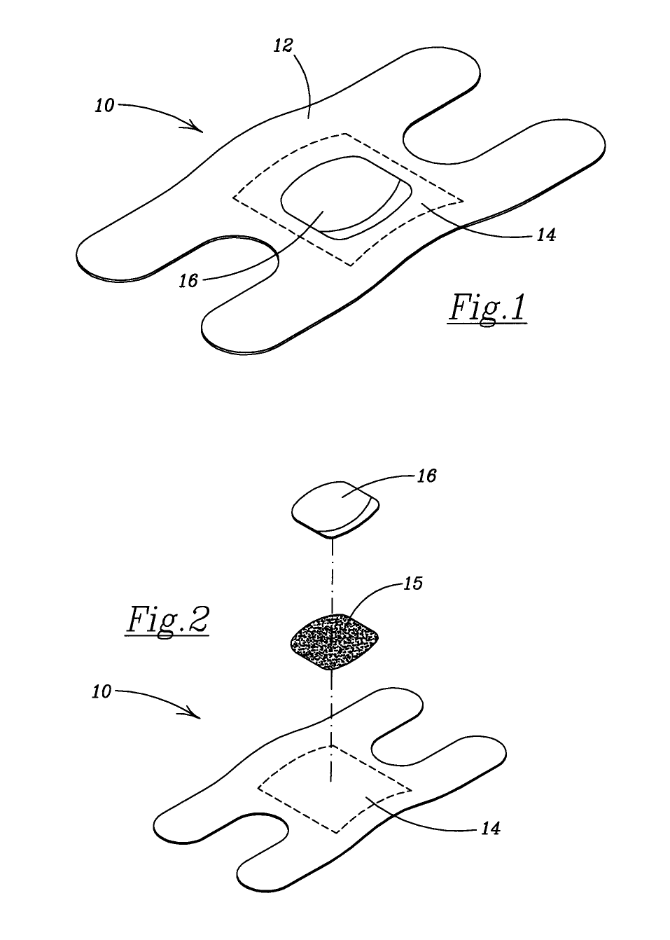

Referring now to the drawings in detail, where like numerals refer to like parts or elements, there is shown in FIG. 1 the combined wound dressing with protective reinforcement section 10 of the present invention. The attachment portion 12 of the combined bandage 10 is configured in the shape of an “H” where each of the extensions are capable of wrapping around the digit to secure the combination bandage 10 in position about the digit and prevent the bandage from moving. A wound dressing portion 14 (shown in dashed lines) is attached to the surface contacting the digit, overlying the injured nail and nail bed of the digit. On the outer surface of the combined bandage 10 is a reinforcing or protective portion 16 that also overlies the injured nail and nail bed and provides a hardened surface to prevent further injury to the nail or nail bed. As shown, the protective portion 16 is colored to represent a finger or toe nail and is dimensioned to completely overlie and extend slightly beyond the nail and nail bed of the affected digit.

The combined bandage 10 is constructed as shown in FIG. 2. Attached to the underside of the digit attachment portion 12 is the wound dressing portion 14, which is centered with respect to the length and width of the attachment portion 12. On the outer surface of the attachment portion 12 an adhesive pad 15 is centered with respect to the length and width of the attachment portion 12 and adhered to it. On top of the adhesive pad 15 the protective portion 16 is adhered completing the structure of the combined bandage 10.

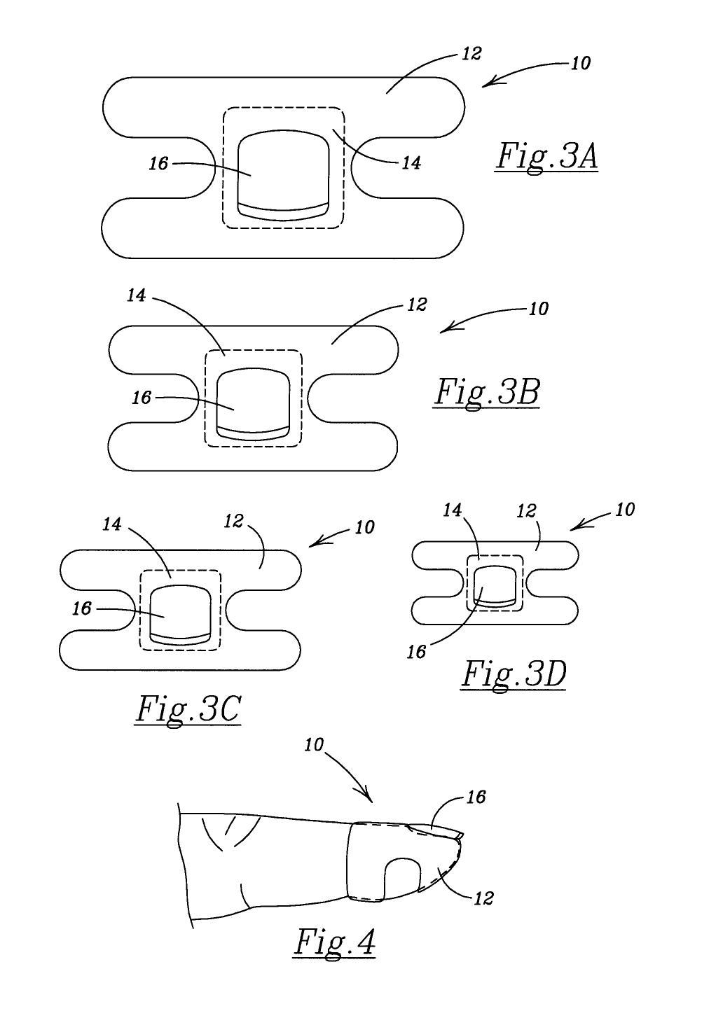

The combined bandage 10 is preferred, due to dimensional measurements of differently sized digits for people of different sizes and shapes, to be of several different sizes. Referring to FIGS. 3A-3D, the combined bandage is sized in extra-large, large, medium and small sizes, respectively. The extra-large size (FIG. 3A) is suggested for use in very large individuals or with thumbs and large toes that a large size combined bandage will not quite fit. The large size combined bandage (FIG. 3B) is dimensioned to be used for adults having larger hands and feet for the larger of the digits. The medium size combined bandage (FIG. 3C) is dimensioned for smaller adults, teenagers and children who have not yet achieved full growth. The small size combined bandage (FIG. 3D) is used on smaller digits for smaller adults, teenagers and children as well. The size of the several portions 12, 14 and 16 of the combined bandage 10 are dimensioned smaller for each succeeding smaller size from extra-large to small as shown to be capable of accommodating very large to very small digits.

In use, the combined bandage 10 is wrapped about a finger, as shown in FIG. 4, but may be any digit, finger or toe of a subject. The extensions of the “H” shaped attachment portion 12 circumscribe the finger from top to bottom overlapping on the inside of the finger. The protective portion 16 directly overlies the injured nail and nail bed of the finger. In use the protective portion 16 is configured to look like a natural finger nail with similar colorations.

Since the protective portion 16 provides sufficient reinforcement to the combined bandage 10 over the injured nail and nail bed, no further splinting of excessive wrapping of wound dressing material or thick bandaging is necessary. The protective portion 16 is dimensioned, with the appropriate sized combined bandage 10, to not only overlie the injured nail and nail bed, but to extend outward in all directions to contact non-injured portions of the digit to provide a solid reinforcing surface to protect the injured nail and nail bed from further injury through direct force, pressure or incidental striking. Without the unnecessary splinting or excessive bandaging of the injured digit, the subject can return to his or her daily routine with substantially complete use of the injured digit.

The present invention may be embodied in other specific forms without departing from the spirit or essential attributes thereof and, accordingly, the described embodiments are to be considered in all respects as being illustrative and not restrictive, with the scope of the invention being indicated by the appended claims, rather than the foregoing detailed description, as indicating the scope of the invention as well as all modifications which may fall within a range of equivalency which are also intended to be embraced therein.

1. A combined bandage comprising:
an attachment portion for overlying and circumscribing a human digit having an injured nail and nail bed configured with a plurality of extensions to wrap around the injured digit and secure said combined bandage in place;
a wound dressing portion attached to and centered on the underside of the attachment portion for contacting the injured nail and nail bed;
an adhesive pad attached to and centered on the top side of the attachment portion capable of receiving and retaining a protective portion; and
a protective portion sized to overlie the injured nail and nail bed adhered to the adhesive pad for providing a solid reinforcing surface to protect the injured nail and nail bed from further injury through direct force, pressure or incidental striking.

2. The combined bandage of claim 1, wherein said protective portion not only overlies the injured nail and nail bed, but is dimensioned to extend outward in all directions to contact non-injured portions of the injured digit.

3. The combined bandage of claim 1, wherein the protective portion is configured and colored to resemble a human finger or toe nail.

4. The combined bandage of claim 1, wherein the overall size of the combined bandage is variable to proportionately fit differently sized human digits.
